(19) United States
(12) Patent Application Publication
(10) Pub. No.: US 2024/0018453 A1
(43) Pub. Date: Jan. 18, 2024

(54) BIOPROCESSING VESSEL HAVING INTEGRAL FLUID CONDUIT

(71) Applicant: GLOBAL LIFE SCIENCES SOLUTIONS USA LLC, MARLBOROUGH, MA (US)

(72) Inventor: HANNA-LEENA SAUKKONEN, BOSTON, MA (US)

(73) Assignee: GLOBAL LIFE SCIENCES SOLUTIONS USA LLC, MARLBOROUGH, MA (US)

(21) Appl. No.: 18/031,664

(22) PCT Filed: Oct. 28, 2021
(86) PCT No.: PCT/US2021/057096

§ 371 Date: Apr. 13, 2023

Related U.S. Application Data
(60) Provisional application No. 63/110,632, filed on Nov. 6, 2020.

(51) Int. Cl. C12M 1/00; C12M 1/21; C12M 1/06; C12M 1/12

(52) U.S. Cl. CPC C12M 23/14; C12M 23/26; C12M 41/02; C12M 23/34; C12M 27/06; C12M 25/02

(57) ABSTRACT

A bioprocessing apparatus includes a flexible bag having an interior volume configured to contain a fluid, and an integral fluid conduit within the flexible bioprocessing bag. The integral fluid conduit includes a panel of material joined to an interior sidewall of the flexible bag so as to define a fluid channel between the interior sidewall of the flexible bag and the panel of material. The integral fluid conduit may include a bottom outlet opening, a top outlet opening, or both. The apparatus may further include a port in a top of the flexible bag, a port in the bottom of the flexible bag, or both, wherein the integral fluid conduit is fluidly connected to the ports.

This Application claims priority to U.S. Provisional Patent Application No. 63/110,632, entitled BIOPROCESSING VESSEL HAVING INTEGRAL FLUID CONDUIT, filed on Nov. 6, 2020, the entirety of which is incorporated by reference.

### TECHNICAL FIELD

Embodiments of the invention relate generally to bioprocessing systems and methods and, more particularly, to a bioprocessing vessel having at least one integral fluid conduit.

BACKGROUND

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel.

Depending on the fluid being processed, the bioreactor system may include a number of fluid lines and different sensors, probes and ports coupled with the bag for monitoring, analytics, sampling, and liquid transfer. For example, a harvest port is typically located at the bottom of the disposable bag and the vessel, and allows for a harvest line to be connected to the bag for harvesting and draining of the bag. In addition, existing bioreactor systems typically utilize spargers for introducing a controlled amount of a specific gas or combination of gases into the bioreactor. A sparger outputs small gas bubbles into a liquid in order to agitate and/or dissolve the gas into the liquid, or for carbon dioxide stripping. The delivery of gas via spargers helps in mixing a substance, maintaining a homogenous environment throughout the interior of the bag, and is sometimes essential for growing cells in a bioreactor. Ideally, the spargers and the agitator are in close proximity to ensure optimal distribution of the gases throughout the container.

Moreover, media additions to the system are typically carried out using J-tubes or dip tubes. A J-tube is a J shaped tubing assembly that extends into the bag from the top, and which has an outlet that faces the interior bag wall. Fluid such as, for example, fresh media, is introduced into the J-tube, and is directed at the interior wall of the bag above the fluid level within the bag. The added fluid travels downwardly along the interior wall via the force of gravity. As illustrated in FIG. 1, however, J-tubes may be prone to misorientation or alignment (e.g., during packaging, transport or unpackaging), where they can turn away from the interior wall. When the tube is not pointed to the wall, a user must manually try to align it to the proper orientation (if misalignment is identified). Otherwise, it can lead to fluid additions falling directly into the fluid within the bioprocessing bag from, which has the potential to cause issues such as cell rupture.

Dip tubes, on the other hand, are long tubes that hang from the top of the bag and extend downwardly toward the bag bottom, through which fluid additions are carried out. As illustrated in FIG. 2, however, such dip tubes can be prone to entanglement with the agitator/impeller which could lead to integrity loss. Such dip tubes may also experience fouling or cleanliness issues due to the fact that they are always in contact with the fluid within the bioreactor bag.

In view of the above, there is a need for a system and method for making fluid additions to a bioprocessing system that are not susceptible to the issues encountered with existing J-tubes or dip tubes.

### BRIEF DESCRIPTION

In an embodiment, a bioprocessing apparatus for the manufacture of biopharmaceutical products comprises: a flexible bag having an interior volume configured to contain a fluid; and an integral fluid conduit within the flexible bioprocessing bag, comprising: a panel of material joined to an interior sidewall of the flexible bag so as to define a fluid channel between the interior sidewall of the flexible bag and the panel of material.

The integral fluid conduit includes a bottom outlet opening, the bottom outlet opening in fluid communication with the interior volume.

The apparatus further comprises: a first port in a top of the flexible bag; wherein a top of the integral fluid conduit is fluidly connected to the first port.

The apparatus further comprises: a second port in a bottom of the flexible bag; wherein a bottom of the integral fluid conduit is fluidly connected to the second port; and wherein integral fluid conduit is not in fluid communication with the interior volume.

The panel of material is an elongated piece of material and is welded, heat sealed, or glued to the interior sidewall of the flexible bag to create opposed vertically extending seals with the interior sidewall, such that a channel is formed between the elongated piece of material and the interior sidewall.

One or both of the opposed vertically extending seals is shorter than a length of the elongated piece of material, creating a flap.

The panel of material includes pores and/or comprises a porous membrane.

The panel of material is at least partially made from or coated with a foam-reducing material.

The fluid conduct is configured to act as a sparger, a filter, a sterile addition tube, a tube holder, a baffle, or a temperature regulating conduit.

The apparatus further comprises at least one tube, the at least one tube being at least partially located within the integral fluid conduit.

In an embodiment, a method for use in manufacture of biopharmaceutical products comprises: providing a flexible bag having an interior volume configured to contain a fluid; and creating an integral fluid conduit within the flexible bioprocessing bag, comprising: joining a panel of material to an interior sidewall of the flexible bag so as to define a fluid channel between the interior sidewall of the flexible bag and the panel of material.

The integral fluid conduit includes a bottom outlet opening, the bottom outlet opening in fluid communication with the interior volume, the method further comprising introducing at least one fluid or gas into the interior volume through the integral fluid conduit and the bottom outlet opening.

The flexible bag includes a first port in a top of the flexible bag; wherein a top of the integral fluid conduit is fluidly connected to the first port; and wherein the at least one fluid or gas is introduced through the first port.

The flexible bag includes a first port in a top of the flexible bag and a second port in a bottom of the flexible bag; wherein a top of the integral fluid conduit is fluidly connected to the first port and a bottom of the integral fluid conduit is fluidly connected to the port such that the fluid conduit is not in fluid communication with the interior volume, the method further comprising introducing a fluid into the integral fluid conduit.

The panel of material is an elongated piece of material, the method comprising welding, heat sealing, or gluing the panel of material to the interior sidewall of the flexible bag to create opposed vertically extending seals with the interior sidewall, such that a channel is formed between the elongated piece of material and the interior sidewall.

One or both of the opposed vertically extending seals is shorter than a length of the elongated piece of material, creating a flap.

The method further comprises including pores and/or a porous membrane on the panel of material.

The panel of material is at least partially made from or coated with a foam-reducing material.

The method further comprises sparging a gas through the integral fluid conduit; filtering at least one component of a fluid using the fluid conduit; or providing at least one tube within the integral fluid conduit.

The method further comprises providing at least one tube, the at least one tube being at least partially located within the integral fluid conduit.

### DRAWINGS

The present invention will be better understood from reading the following description of non-limiting embodiments, with reference to the attached drawings, wherein below:

FIG. 1 is a simplified side elevational, cross-sectional view of a prior art bioreactor system, illustrating use of a J-tube.

FIG. 2 is a simplified side elevational, cross-sectional view of a prior art bioreactor system, illustrating use of a dip tube.

FIG. 3 is a front elevational view of a bioreactor system according to an embodiment of the invention.

FIG. 4 is a simplified side elevational, cross-sectional view of the bioreactor system of FIG. 3.

FIG. 5 is a simplified side elevational, cross-sectional view of a bioprocessing bag having integral fluid channels according to an embodiment of the invention, for use with the system of FIG. 3.

FIG. 6 is a simplified side elevational, cross-sectional view of a bioprocessing bag having integral fluid channels according to another embodiment of the invention, for use with the system of FIG. 3.

FIG. 7 is a simplified, top cross-sectional view of the bioprocessing bag of with at least one integral fluid channel of FIG. 5 or 6.

FIG. 8 is an enlarged, detail view of a portion of the bioprocessing bag of FIG. 5 or 6 illustrating a port and external tubing connected to at least one integral fluid channel.

FIG. 9 is a simplified side elevational, cross-sectional view of a bioprocessing bag having integral fluid channels according to another embodiment of the invention, for use with the system of FIG. 3

FIG. 10 is a simplified side elevational, cross-sectional view of a bioprocessing bag having integral fluid channels according to further embodiments of the invention, for use with the system of FIG. 3

FIGS. 10A-C illustrate multiple simplified side elevational, cross-sectional views of a bioprocessing bag having integral fluid channels according to further embodiments of the invention, for use with the system of FIG. 3

FIGS. 11A-B illustrate multiple simplified side elevational, cross-sectional views of a bioprocessing bag having integral fluid channels according to further embodiments of the invention, for use with the system of FIG. 3

FIG. 12 illustrates a simplified side elevational, cross-sectional view and an angled cross-sectional view of a bioprocessing bag having integral fluid channels according to further embodiments of the invention, for use with the system of FIG. 3

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems. As used herein, the term “bag” means a flexible or semi-rigid container or vessel used, for example, as a bioreactor or mixer for the contents within.

Embodiments of the invention provide an apparatus, system and method for making fluid additions to a bioprocessing system. In one embodiment, a bioprocessing apparatus includes a flexible bag having an interior volume configured to contain a fluid, and an integral fluid conduit within the flexible bioprocessing bag, the integral fluid conduit being configured to deliver a second fluid to the interior volume. The integral fluid conduit includes a panel of material joined to an interior sidewall of the flexible bag so as to define a fluid channel between the interior sidewall of the flexible bag and the panel of material. The integral fluid conduit includes a bottom outlet opening. The apparatus further includes a port in a top of the flexible bag, wherein a top of the integral fluid conduit is fluidly connected to the port.

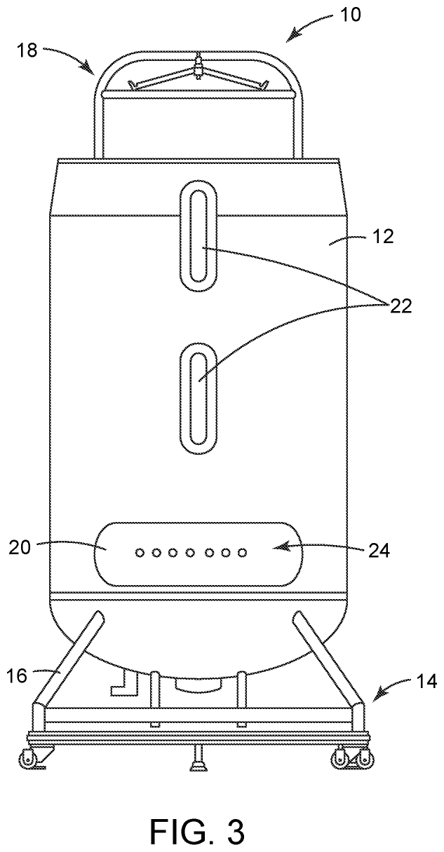

With reference to FIG. 3, a bioreactor or bioprocessing system 10 according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000 L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO²), mixing rate, nutrients, foam, and gas flow rate, for example.

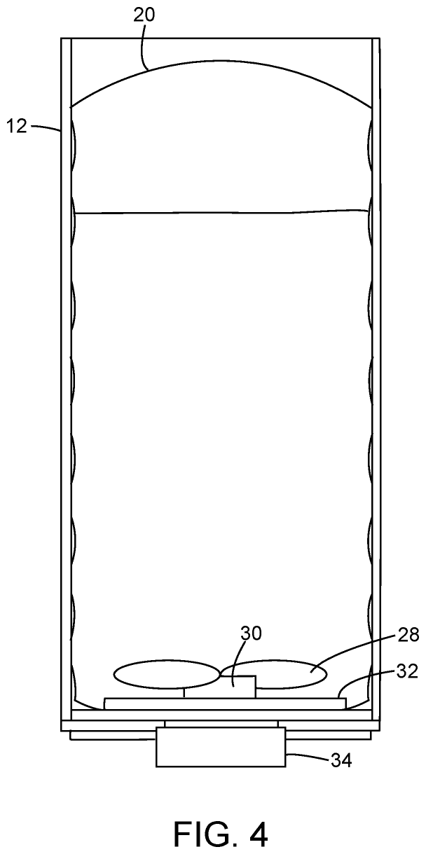

With specific reference to FIG. 4, a schematic side elevational, cutaway view of the bioreactor system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem TM, Bioclear™ 10 and Bioclear 11 laminates, available from GE Healthcare Life Sciences. Portions of the flexible container can comprise a substantially rigid material such as a rigid polymer, for example, high density polyethylene, metal, or glass. The flexible bag may be supplied pre-sterilized, such as using gamma irradiation.

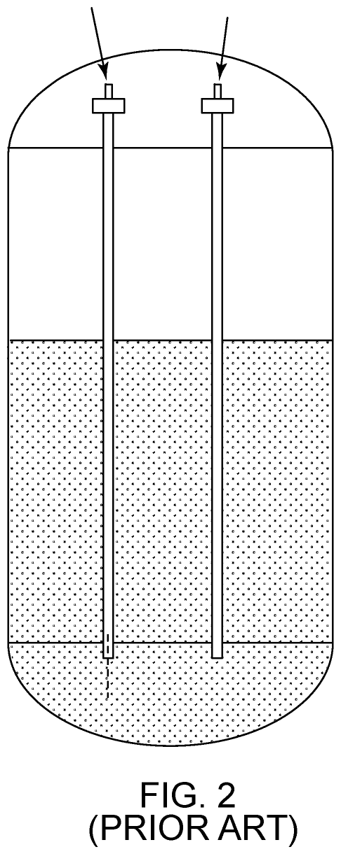

The flexible bag 20 contains an impeller 28 attached to a magnetic hub 30 at the bottom center of the inside of the bag, which rotates on an impeller plate (not shown) also positioned on the inside bottom of the bag 20. Together, the impeller 28 and hub 30 (and in some embodiments, the impeller plate) form an impeller assembly. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven impellers.

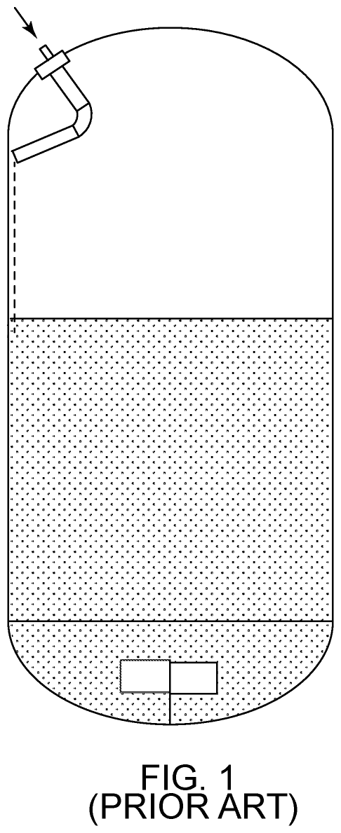

As also illustrated in FIG. 4, the flexible bag 20 contains a sparger device 32 that can be engaged with a port (not shown) on the bottom of the flexible bag 20, which receives a supply of gas from a gas supply line. The sparger device 32, as is known in the art, is configured to introduce gas bubbles into the culture/media within the flexible bag 20. While FIGS. 1 and 2 illustrate the bioreactor vessel 12 as containing a single use, flexible bag 20, it is contemplated that the flexible bag 20 may be omitted in which case the bioprocessing/culturing operations can take place directly within the vessel 12.

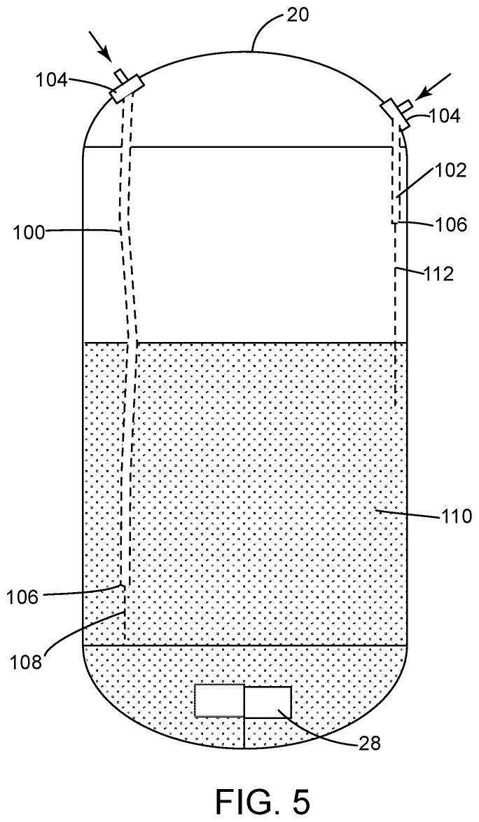

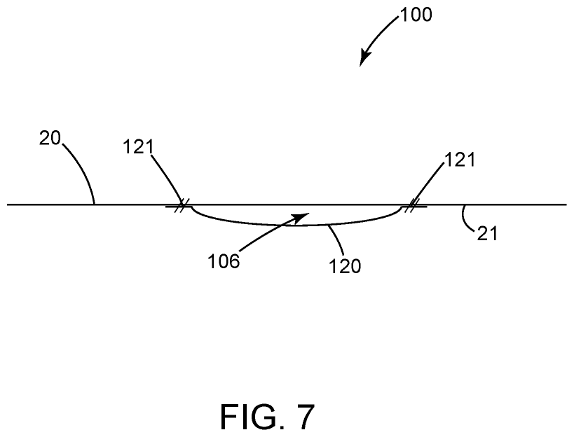

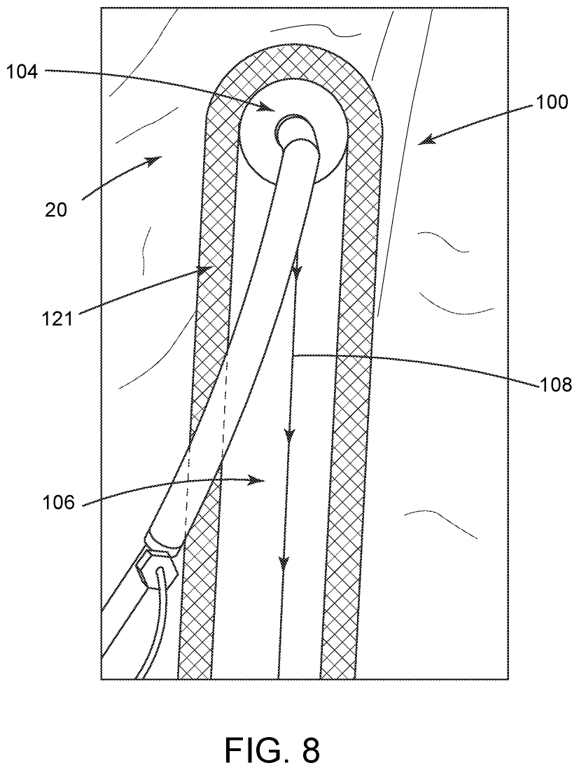

With reference to FIGS. 5-12, the flexible bioprocessing bag 20 may have one or more integral fluid conduits or channels 100, 102 through which a fluid may be added or introduced into the bag 20. As shown in FIG. 5, the fluid conduits 100, 102 are integral with the interior sidewall of the bag 20. For example, in an embodiment, the fluid conduits 100, 102 may be formed by welding, heat sealing, adhering, or otherwise securing an elongated piece of material 120 to the interior sidewall 21 of the bag 20 to create opposed vertically extending seals 121 with the interior wall 21, such that a channel 106 is formed between the elongated piece of material and the interior sidewall 21 (See, e.g., FIG. 7). In an embodiment, the material 120 is a fluid compatible material such as polyethylene or Fortem, although other materials known in the art may also be utilized without departing from the broader aspects of the invention. As illustrated in FIGS. 5 and 8, a top of each integral conduit 100, 102 may be fluidly connected to a respective port 104 adjacent to, and accessible from, a top of the bag 20, while a bottom of each conduit 100, 102 may have an outlet opening 106 in fluid communication with the interior of the bag 20 (i.e., there is no seal with the interior wall of the bag at the bottom of the conduit 100, 102). As also shown in FIG. 8, the material 120 is also adhered around the top of the port (e.g., in a semicircular shape).

In an embodiment, the fluid conduit 100 may extend from the top of the bag 20 to a point adjacent to the bottom of the bag 20 so as to allow for the introduction of a fluid 108 directly into a volume of fluid 110 within the bag 20 using port 104. Alternatively, or in addition, the bag 20 may be provided with integral fluid conduit 102 that extends to a vertical location above the level of fluid 110 within the bag 20. In such case, fluid introduced through port 104 may exit the outlet 106 of conduit 102 at a point above the fluid level within the bag. As the conduit 102 is integrally formed with the interior bag wall, the fluid 110 will, once exiting through outlet 106, and via surface tension and/or adhesion, trickle down the interior bag wall under the force of gravity and into the volume of fluid 110 within the bag 20.

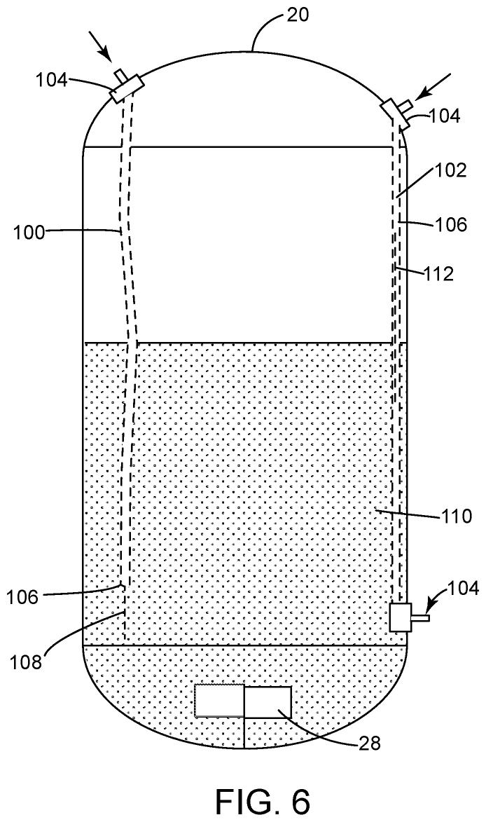

According to an embodiment as illustrated in FIG. 6, the flexible bioprocessing bag 20 may have one or more integral fluid conduits or channels 100, 102 through which a fluid or gas is introduced or circulated. As shown in FIG. 6, the fluid conduits 100, 102 are integral with the interior sidewall of the bag 20, in the same way as described with regard to FIG. 5. As illustrated in FIGS. 6 and 8, a top of each integral conduit 100, 102 may be fluidly connected to a respective first port 104 adjacent to, and accessible from, a top of the bag 20, while a bottom of each conduit 100, 102 may have an outlet connected to a second port 104 located at the bottom of the bag. As also shown in FIG. 8, the material 120 is also adhered around the top of the first port (e.g., in a semicircular shape) and around a bottom of the second port (not shown). In such a configuration, for example, the integrated fluid conduits 100, 102 can act as a temperature regulating element for the bag 20. Specifically, by circulating a heated or cooled gas or fluid through the integrated fluid conduits 100, 102 the temperature of the contents of the bag 20 can be raised or lowered, respectively. In this way, the integrated fluid conduits 100, 102 can replace existing thermal jackets that are used to maintain the temperature of the bag 20 during bioprocessing, as will be further described below.

In a still further embodiment, the flexible bioprocessing bag 20 may have one or more integral fluid conduits or channels 100, 102 through which a fluid or gas is introduced or circulated. The fluid conduits 100, 102 are integral with the interior sidewall of the bag 20, in the same way as described with regard to FIG. 5. A bottom of each conduit 100, 102 may have an outlet connected to a second port 104 located at the bottom of the bag, while a top of each conduit 100, 102 may have an outlet opening 106 in fluid communication with the interior of the bag 20 (i.e., there is no seal with the interior wall of the bag at the bottom of the conduit 100, 102).

In all embodiments, the length of the fluid conduits 100, 102 can be selected such that addition of liquids can be targeted to specific locations within the bag. For example, a length can be selected such that the outlet opening 106 corresponds to a liquid height of liquid within the interior space of the bag 20 (e.g., for application of a substance, such as an antifoam agent) to the liquid surface). As another example, the length can be selected such that the outlet opening 106 are set to a target liquid volume level of the bag for harvest or withdrawal of a volume in the bag 20, thus leaving a known volume within the bag 20. This advantageously allows for the bag to act as a continuous seed vessel by removing excess volume and refeeding the “heel” (i.e., remainder) fluid.

In an embodiment, the length of the conduits 100, 102, and the location of the first and/or second port 104 may be selected so as to introduce fluid into the bag 20 at any vertical location desired. Moreover, it is contemplated that any number of integral conduits may be employed to allow for any number of fluid introduction locations desired. In an embodiment, it is contemplated that the integral fluid conduits 100, 102 of the invention may be utilized to introduce fresh media into the bag 20, although the invention is not intended to be so limited in this regard. In particular, it is contemplated that the integral conduits 100, 102 of the invention may be utilized to introduce or remove other fluids, substances or compositions into the bag 20, such as, for example, cells and the like. In yet other embodiments, it is contemplated that the integral conduits can also be used to remove fluid (e.g., media, cells, or air/gas from the bag 20), by connecting the ports 104 to a suction source (e.g., pump) or other evacuation means. In still further embodiments, the integral fluid conduits 100, 102 are connect to a bottom wall of the bag 20. For example, the integral fluid conduits 100, 102 can be located along a portion of the bottom of bag 20 such that one end of the integral fluid conduits 100, 102 is connected to a port 104 in the bottom or sidewall of the bag at a periphery of the bag, while the channel 106 is generally located in the center of the of bottom of the bag (e.g., underneath and/or near the location of impeller 28. Moreover, while bag 20 has been disclosed herein as being a disposable, flexible bioprocessing bag, the invention is not so limited in this regard. In particular, it is contemplated that the integral conduits of the invention may be incorporated into or utilized with rigid bioreactor vessels as well (e.g., rigid vessel 12).

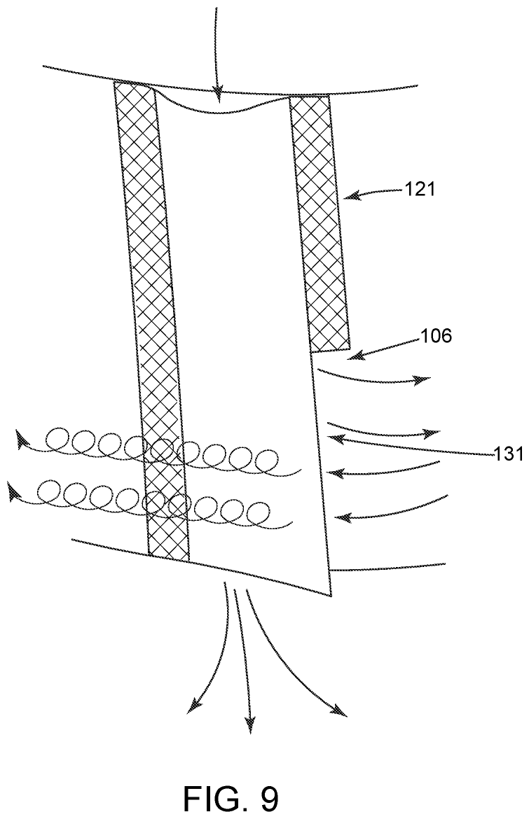

With further reference to FIG. 9, the panel of material is an elongated piece of material and is welded, heat sealed, or glued to the interior sidewall of the flexible bag to create opposed vertically extending seals 121 with the interior sidewall, such that a channel is formed between the elongated piece of material and the interior sidewall. In an embodiment, one or both of the opposed vertically extending seals 121 is shorter than a length of the elongated piece of material, creating a flap 131. By creating a flap at the location of the outlet, turbulent flow is promoted (as illustrated by the arrows), aiding in the mixing of the fluid or gas introduced through the integral conduits 100, 102 with fluid with the bag 20. The illustrated flap 131 is merely one configuration that can be used to aid in mixing. Additional shapes and sealing locations (e.g., the flap could have an arced or angled shape) are within the scope of the invention.

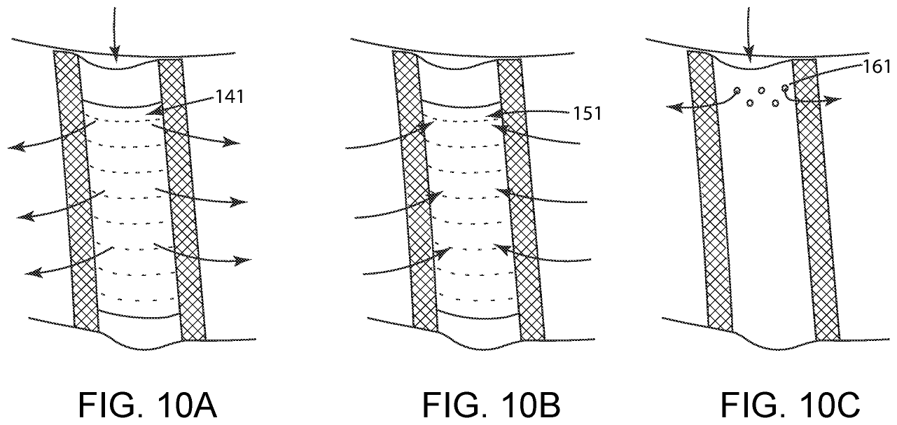

With reference to FIGS. 10A-C, the integral conduits 100, 102 may also be formed with, or include pores and/or a porous membrane. As FIGS. 10A-C illustrates, at least a section of the integral conduits 100, 102 may be made from a porous membrane or include an array of pores 141. With specific reference to FIG. 10A, fluid can be sterilely introduced through the integral conduits 100, 102 and into the inner volume of the bag. Specifically, the pore size can be selected such that potential contaminants located in the introduced fluid are unable to pass through the pores, while also maintaining a pore size that prevents the cells from entering the integral conduits 100, 102. Further, as FIG. 10B illustrates, at least a section of the integral conduits 100, 102 may be made from a porous membrane or include an array of pores 151 such that the integral fluid conduits 100, 102 act as a filter. Specifically, the pore size can be selected such that waste material within the fluid in the bag 20 is capable of passing through the pores. The pore size can also be selected such that the filter acts as a depth filter for cell removal. Still further, as FIG. 10C illustrates, the at least a section of the integral conduits 100, 102 may be made from a porous membrane or include an array of pores 161 such that the integral fluid conduits 100, 102 act as a sparger. For example, an array of pores/porous membrane 161 can have a pore size selected such that a gas introduced into the integral fluid conduits 100, 102 exits through the pores/porous membrane 161 and bubbles into the fluid located within the bag 20. As mentioned above, the integral conduits 100, 102 can run along the bottom of the bag 20, such that the pores/porous membrane 161 and/or channel 106 can be located near the center of the bottom of bag such that the introduced gas is mixed by the sparger 28.

With further reference to FIGS. 10A-C, and as discussed above, the integral fluid conduits 100, 102 may be connected to a top and/or a bottom port 104. Depending upon the specific application and user requirements, one end of the integral fluid conduits 100, 102 may be sealed (as opposed to being open into the interior volume of the bag or connected to a port 104). By way of example only, when the integral fluid conduits 100, 102 are used for the sterile addition of materials into the inner volume of bag 20, the top of the integral fluid conduits 100, 102 may be connected to a top port 104, while the bottom of integral fluid conduits 100, 102 is sealed to the interior sidewall 21. Such a configuration ensures that material entering the interior volume of bag 20 only passes through the array of pores 161. Similarly, when the integral fluid conduits 100, 102 are used for filtration of materials out of the inner volume of bag 20, the bottom of the integral fluid conduits 100, 102 may be connected to a bottom port 104, while the top of integral fluid conduits 100, 102 is sealed to the interior sidewall 21. Such a configuration allows gravity to aid in filtration. When the integral fluid conduits 100, 102 are used for sparging into the inner volume of bag 20 either configuration may be implemented (i.e., the top of the integral fluid conduits 100, 102 is connected to a top port 104 with the bottom of the integral fluid conduits 100, 102 being sealed to the interior sidewall 21 or vis versa. Such a configuration ensures that gas only exits through pores 161.

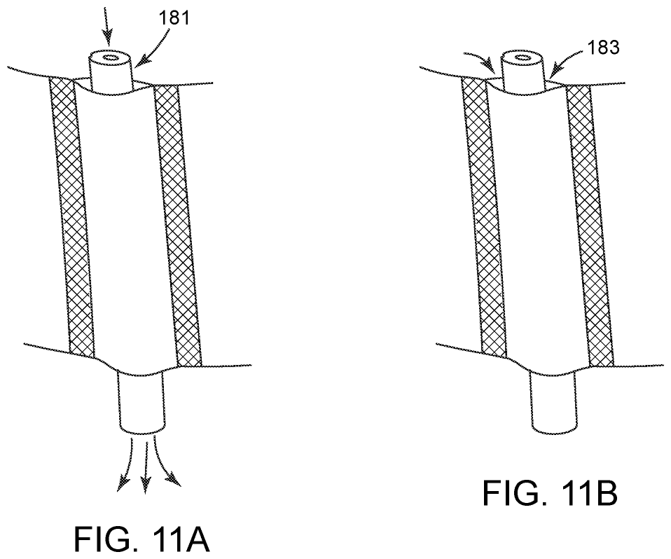

With reference to FIGS. 11A-B, integral fluid conduits 100, 102 may also be configured to act as a tube anchor. For example, since tubes, such as tube 181 (e.g., dip tube, exhaust tube, etc.) have known issues with misalignment when in use, the integral fluid conduits 100, 102 can be used to anchor tube 181 to the interior sidewall 21, thus ensuring that they cannot substantially move and become misaligned. Additionally, fluid can be passed through integral fluid conduits 100, 102 to help regulate the temperature of the tube 181. For example, when tube 181 is an exhaust tube, a cooling fluid can be passed into integral fluid conduits 100, 102 such that the temperature of the exhaust line and the gases and/or fluids passing therethrough are cooled, thereby condensing moisture within the exhaust line. Similarly, if the fluid/gas passing through tube 181 needs to be heated a heated fluid can be passed into integral fluid conduits 100, 102 such that the temperature of the tube 181 is increased.

Moreover, instead of acting as a tube anchor, the integral fluid conduits 100, 102 can act as a channel for placement of probes within the bag 20. For example, typical bioreactors include at least one port for insertion of a probe sensor to measure, for example, pH, DO, CO2 concentration, etc. The integral fluid conduits 100, 102 can provide an easy and cost-effective way to place sensors within a fluid in the bag 20, and is not limited by the need to have the probe port generally located at a position where the probe is introduced into the bag 20.

While the embodiments depicted in FIGS. 11A-B illustrate integral fluid conduits 100, 102 being open at the top and bottom (e.g., the integral fluid conduits 100, 102 are not sealed to the interior wall 2), the invention is not so limited. For example, the integral fluid conduits 100, 102 can have their top and bottom connected to ports (e.g., ports 104), the ports also being in fluid communication with tube 181. Additionally, while only one tube 181 is illustrated as being held within the integral fluid conduits 100, 102, more than one tube 181 can be located in a given integral fluid conduit (e.g., a bundle of tubes 181 can be restrained by one integrated fluid conduit).

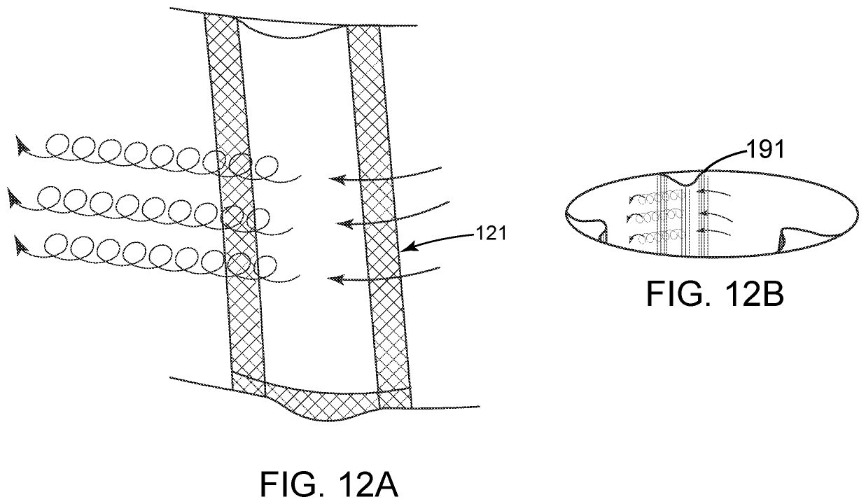

With reference to FIG. 12, the integrated fluid conduits 100, 102 can also act as a baffle 191 within the bag 20, according to an embodiment. For example, the integrated fluid conduits 100, 102 can be filled with a pressurized liquid or gas, or filled with a solid material, such that the integrated fluid conduits 100, 102 create baffles along the length of the bag 20. Such baffles 191 can aid in the mixing of fluid within bag 20 by creating turbulent areas (e.g., depicted by the arrows) around the circumference of the bag 20. In this embodiment, the integrated fluid conduits 100, 102 can be sealed at one end (i.e., the top or bottom end) such that the pressurized gas or liquid enters one end of the integrated fluid conduits 100, 102 (e.g., through a port 104). Alternatively, both ends of the integrated fluid conduits 100, 102 can be connected to ports, and the pressurized gas or liquid can be circulated through the integrated fluid conduits 100, 102.

In any of the embodiments described herein, the integral fluid conduits 100, 102 may at least partially made from or coated with a foam-reducing material. Since the integral fluid conduits 100, 102 are in direct contact with the fluid and/or headspace of the bag 20, the foam reducing material makes direct contact with foam created during the bioprocess and can mitigate foam collecting in the head space, which is known to clog/foul filters (e.g., exhaust filters).

Additionally, the integrated fluid conduits 100, 102 can be connected to pump(s) in order provide the necessary motive force to move gases and/or fluids through the integrated fluid conduits 100, 102. For example, the pump(s) can be operated to provide an array of flow rates of fluids through the integrated fluid conduits 100, 102, depending upon the specific application the integrated fluid conduits 100, 102 are being used for.

The integrated fluid conduits 100, 102 of the present invention, and all embodiments thereof, can be created in any number of shapes, and not just as linear conduits. For example, the integrated fluid conduits 100, 102 can be formed into serpentine, arcuate, angular, and other shapes. Additionally, the integrated fluid conduits 100, 102 do not have to extend from in the direction from the top of the bag 20 to the bottom. The integrated fluid conduits 100, 102 could extend at any angle or generally extend in a circumferential direction around the bag 20.

The integrated fluid conduits 100, 102 of the present invention, and all embodiments thereof, can be made from rigid as well as flexible materials.

The bioprocessing bag having integral fluid conduits disclosed herein obviates issues with misalignment, which has heretofore been an issue with the use of J-tubes and dip tubes, as well as eliminates the possibility of the fluid conduit mechanically interfering with the impeller (which can be the case with the use of dip tubes). Moreover, another major advantage of the integral fluid conduits disclosed herein is that they can be sterilized with the bioreactor bag, providing an aseptic fluid channel that does not require separate sterilization.

While the above-described embodiments relate to integral fluid conduits that are located on the inside of the bag 20, it is also envisioned that the integral fluid conduits could be located on an external surface of bag 20. In such a configuration, the integral fluid conduits can provide an easy way to route tubes, wires, etc. that are connected to, or otherwise located adjacent the bioreactor.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A bioprocessing apparatus for the manufacture of biopharmaceutical products, comprising:
a flexible bag having an interior volume configured to contain a fluid; and
an integral fluid conduit within the flexible bioprocessing bag, comprising:
a panel of material joined to an interior sidewall of the flexible bag so as to define a fluid channel between the interior sidewall of the flexible bag and the panel of material.

2. The bioprocessing apparatus of claim 1, wherein:
the integral fluid conduit includes a bottom outlet opening, the bottom outlet opening in fluid communication with the interior volume.

3. The bioprocessing apparatus of claim 14ar_2, further comprising:
a first port in a top of the flexible bag;
wherein a top of the integral fluid conduit is fluidly connected to the first port.

4. The bioprocessing apparatus of claim 14ar-3, further comprising:
a second port in a bottom of the flexible bag;
wherein a bottom of the integral fluid conduit is fluidly connected to the second port; and
wherein integral fluid conduit is not in fluid communication with the interior volume.

5. The bioprocessing apparatus of claim 1, wherein the panel of material is an elongated piece of material and is welded, heat sealed, or glued to the interior sidewall of the flexible bag to create opposed vertically extending seals with the interior sidewall, such that a channel is formed between the elongated piece of material and the interior sidewall.

6. The bioprocessing apparatus of claim 5, wherein one or both of the opposed vertically extending seals is shorter than a length of the elongated piece of material, creating a flap.

7. The bioprocessing apparatus of claim 1, wherein the panel of material includes pores and/or comprises a porous membrane.

8. The bioprocessing apparatus of claim 1, wherein the panel of material is at least partially made from or coated with a foam-reducing material.

9. The bioprocessing apparatus of claim 1, wherein the fluid conduct is configured to act as a sparger, a filter, a sterile addition tube, a tube holder, a baffle, or a temperature regulating conduit.

10. The bioprocessing apparatus of claim 1, further comprising at least one tube, the at least one tube being at least partially located within the integral fluid conduit.

11. A method for use in manufacture of biopharmaceutical products, comprising:
providing a flexible bag having an interior volume configured to contain a fluid; and
creating an integral fluid conduit within the flexible bioprocessing bag, comprising:
joining a panel of material to an interior sidewall of the flexible bag so as to define a fluid channel between the interior sidewall of the flexible bag and the panel of material.

12. The method of claim 11,
wherein the integral fluid conduit includes a bottom outlet opening, the bottom outlet opening in fluid communication with the interior volume, the method further comprising introducing at least one fluid or gas into the interior volume through the integral fluid conduit and the bottom outlet opening.

13. The method of claim 12,
wherein the flexible bag includes a first port in a top of the flexible bag;
wherein a top of the integral fluid conduit is fluidly connected to the first port; and
wherein the at least one fluid or gas is introduced through the first port.

14. The method of claim 11,
wherein the flexible bag includes a first port in a top of the flexible bag and a second port in a bottom of the flexible bag;
wherein a top of the integral fluid conduit is fluidly connected to the first port and a bottom of the integral fluid conduit is fluidly connected to the port such that the fluid conduit is not in fluid communication with the interior volume, the method further comprising introducing a fluid into the integral fluid conduit.

15. The method of claim 11,
wherein the panel of material is an elongated piece of material, the method comprising welding, heat sealing, or gluing the panel of material to the interior sidewall of the flexible bag to create opposed vertically extending seals with the interior sidewall, such that a channel is formed between the elongated piece of material and the interior sidewall.

16. The method of claim 15, wherein one or both of the opposed vertically extending seals is shorter than a length of the elongated piece of material, creating a flap.

17. The method of claim 11, further comprising including includes pores and/or a porous membrane on the panel of material.

18. The method of claim 11, wherein the panel of material is at least partially made from or coated with a foam-reducing material.

19. The method of claim 11, further comprising:
sparging a gas through the integral fluid conduit;
filtering at least one component of a fluid using the fluid conduit; or
providing at least one tube within the integral fluid conduit.

20. The method of claim 11, further comprising providing at least one tube, the at least one tube being at least partially located within the integral fluid conduit.
